(19) United States
(12) Patent Application Publication
(10) Pub. No.: US 2019/0345194 A1
(43) Pub. Date: Nov. 14, 2019

(54) METHOD FOR PURIFYING ANTIBODY

(71) Applicant: JNC CORPORATION, Tokyo (JP)

(72) Inventor: YOSHIHIRO MATSUMOTO, Kanagawa (JP)

(73) Assignee: JNC CORPORATION, Tokyo (JP)

(21) Appl. No.: 16/349,617

(22) PCT Filed: Nov. 10, 2017
(86) PCT No.: PCT/JP2017/040599

§ 371 Date: May 14, 2019

(30) Foreign Application Priority Data
Nov. 18, 2016 (JP) 2016-224782

(51) Int. Cl. C07K 1/22; B01J 20/281; B01D 15/38; G01N 30/26; C07K 16/06; G01N 30/88

(52) U.S. Cl. CPC C07K 1/22; B01J 20/281; G01N 30/88; G01N 30/26; C07K 16/065; B01D 15/38

(57) ABSTRACT

An antibody purification method, including bringing a solution containing an antibody and HCP into contact with a chromatography media to separate the antibody and the HCP, wherein the chromatography media contains a base media containing porous particles, and a compound having a plurality of primary amino groups bonded to the base media, and 20 to 55% of the primary amino groups in the compound having the plurality of primary amino groups are modified with a hydrophobic group, and collection efficiency of the antibody is 85% or more, and an amount of the HCP in an antibody solution after purification is less than 45 ppm.

### TECHNICAL FIELD

The invention relates to an antibody purification method. More specifically, the invention relates to an antibody purification method in which an antibody and impurities are separated from a solution containing the antibody and the impurities such as host cell protein (HCP).

### BACKGROUND ART

Purification of a biopharmaceutical or the like by using chromatography is widely known, and an object and impurities are separated by utilizing various intermolecular interactions. Specific examples include ion exchange chromatography utilizing an electrostatic interaction, hydrophobic chromatography utilizing a hydrophobic interaction, and protein A chromatography utilizing an affinity interaction with an antibody.

A technique most frequently applied in purification of the biopharmaceutical is the ion exchange chromatography. In the ion exchange chromatography, adsorbability is well known to decrease by an increase in electrical conductivity of a treatment liquid. Accordingly, in a sample having high electrical conductivity, the electrical conductivity needs to be decreased to 5 mS/cm by dilution or demineralization before applying adsorption treatment thereto, for example.

In order to compensate for such a disadvantage of the ion exchange chromatography, a chromatography media simultaneously having the hydrophobic interaction, a hydrophilic interaction, a chelate interaction and so forth, in addition to the electrostatic interaction, has been actively developed in recent years.

Thus, as the chromatography media simultaneously having a plurality of actions, such a media is commercially available as Capto (registered trademark) adhere, Capto (registered trademark) MMC (both, made by GE Healthcare Ltd.), MEP HyperCel, HyperCel (trademark) AX STAR (all, made by Pall Corporation), Eshumuno (registered trademark) HCX (made by EMD Millipore Corporation), CHT (registered trademark) Ceramic Hydroxyapatite (made by Bio-Rad Laboratories, Inc.) and Toyopearl (registered trademark) MX Trp-650 (made by TOSOH Corporation).

The above chromatography media simultaneously having the plurality of actions is obtained by introducing into an identical base media a ligand having an interaction according to a different principle, in addition to a ligand having the electrostatic interaction, or further modifying a part of the ligand bonded to the base media. The thus obtained media has selectivity different from the selectivity of the ion exchange chromatography media supporting only the ligand having the electrostatic interaction. As the ligand to be bonded to the base media, polyamine is examined, for example.

Patent literature No. 1 discloses a chromatography media having polyamine as a ligand, and use thereof for purification of a blood coagulation factor.

Patent literature No. 2 discloses a porous adsorption medium having a surface covered with a crosslinked polymer of polyallylamine.

Patent literature No. 3 discloses a chromatograph media obtained by bonding polyallylamine to a base media and modifying the polyallylamine with a further functional group, in which a cation exchange group is used as the further functional group.

Patent literature No. 5 describes that a media to which a ligand containing a plurality of kinds of functional groups having different interactions is added can be preferably used for purification of an immunoglobulin.

Patent literature No. 5 suggests that, particularly when a ligand structure having an amino group and a phenyl group is used, the immunoglobulin can be collected by a flow-through fraction at pH 7.

Patent literature No. 6 describes a method in which Sperosil QMA known as a hydrophobic anion exchange media is used to separate an immunoglobulin existing in colostrum or serum thereof by flow-through. Moreover, Patent literature No. 7 describes an example in which a dye binding type chromatography media separates an immunoglobulin derived from cone fraction II from other impurities by flow-through.

Patent literature No. 8 discloses a method in which a media having an anion exchange group and an aromatic group is used in a flow-through mode to purify an antibody.

Non-Patent literature No. 1 describes that a chromatography media obtained by bonding polyethyleneimine to a Sepharose FF resin being a base media, and further modifying the polyethyleneimine with a benzoyl group develops excellent performance in purification of bovine serum albumin (BSA).

As described above, various chromatography medias have been proposed, and development of a chromatography media having adsorption and/or separation characteristics, such as adsorption power, a pore size and a specific surface area suitable for a material to be purified according to a material to be purified is everlasting challenges.

### CITATION LIST

### Patent Literature

Patent literature No. 1: JP H4-506030 A
Patent literature No. 2: JP 2009-53191 A
Patent literature No. 3: JP 2004-522479 A
Patent literature No. 4: JP 2016-6410 A
Patent literature No. 5: JP 2001-501595 A
Patent literature No. 6: U.S. Pat. No. 4,582,580 B
Patent literature No. 7: AU 594054 A
Patent literature No. 8: JP 5064225 B

### Non-Patent Literature

Non-patent literature No. 1: Journal of Chromatography A, 1372 (2014), 157-165

### SUMMARY OF INVENTION

### Technical Problem

Under a background as described above, a chromatography media having adsorption and/or separation characteristics suitable for a material to be purified is required.

### Solution to Problem

The present inventors have diligently continued to conduct study in order to solve the problems described above, and as a result, have found out that a chromatography media obtained by adding polyamine to a base media containing porous particles and then modifying amino groups in the polyamine with a hydrophobic group has excellent adsorbability of protein and can be used for separation and purification of protein (Patent literature No. 4). The present inventors have further progressed study, and thus have found out that a chromatography media obtained by adding a compound having a plurality of primary amino groups to a base media containing porous particles and then modifying a part of the primary amino groups with a hydrophobic group has characteristics suitable particularly for separation and purification of an antibody, and have reached the invention. More specifically, the invention is as described below, for example.

Item 1. An antibody purification method, comprising bringing a solution containing an antibody and host cell protein (HCP) into contact with a chromatography media to separate the antibody and the HCP, wherein the chromatography media contains a base media containing porous particles, and a compound having a plurality of primary amino groups bonded to the base media, and 20 to 55% of the primary amino groups in the compound having the plurality of primary amino groups are modified with a hydrophobic group, and collection efficiency of the antibody is 85% or more, and an amount of the HCP in an antibody solution after purification is less than 45 ppm.

Item 2. The method according to item 1, wherein the method is performed in a flow-through mode.

Item 3. The method according to item 1 or 2, wherein more than 40% of primary amino groups in the compound having the plurality of primary amino groups are modified with the hydrophobic group.

Item 4. The method according to any one of items 1 to 3, wherein the compound having the plurality of primary amino groups is selected from the group of polyallylamine, polyvinylamine, chitosan, polylysine, polyguanidine and polyornithine.

Item 5. The method according to item 4, wherein the compound having the plurality of primary amino groups is polyallylamine.

Item 6. The method according to item 5, wherein weight average molecular weight of the polyallylamine is 5,000 to 15,000.

Item 7. The method according to any one of items 1 to 6, wherein the hydrophobic group has any structure represented by general formulas (1) to (3):

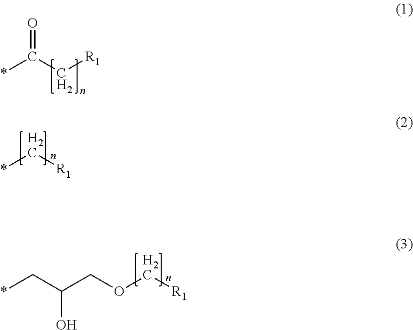

wherein,

n is an integer from 0 to 8,

R₁ is a phenyl group when n is an integer from 0 to 3, and H or a phenyl group when n is an integer from 4 to 8, and

an asterisk (*) is a bonding site with the primary amino group in the compound having the plurality of primary amino groups.

Item 8. The method according to item 7, wherein the hydrophobic group has a structure represented by general formula (1).

Item 9. The method according to item 8, wherein, in general formula (1), n is an integer from 4 to 8, and R₁ is H.

Item 10. The method according to item 8, wherein, in general formula (1), n is an integer from 0 to 8, and R₁ is a phenyl group.

Item 10-1. The method according to item 7, wherein n is an integer from 4 to 8, and R₁ is H, and more than 40% to 55% of the primary amino groups in the compound having the plurality of primary amino groups are modified with the hydrophobic group.

Item 11. The method according to any one of items 1 to 6, wherein the hydrophobic group is a group derived from a compound selected from the group of valeric anhydride, caproic anhydride, enanthic anhydride, caprylic anhydride, pelargonic anhydride, benzoic anhydride, butyl glycidyl ether and phenyl glycidyl ether.

Item 12. The method according to item 11, wherein the hydrophobic group is a group derived from valeric anhydride or benzoic anhydride.

Item 12-1. The method according to item 8, wherein the compound having the plurality of primary amino groups modified with the hydrophobic group includes a repeating unit represented by general formula (a) and a repeating unit represented by general formula (b):

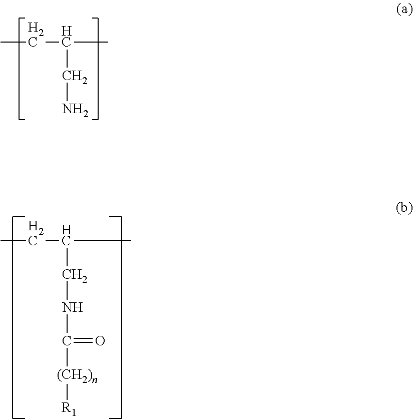

in general formula (b), n and R₁ are as defined in general formula (1).

Item 12-2. The method according to item 12-1, wherein the compound having the plurality of primary amino groups modified with the hydrophobic group has an amino group (—NH₂ group in general formula (a)) being a hydrophilic group and having an electrostatic interaction, an amide group (—NH—CO— group in general formula (b)) having an electrostatic interaction, and a hydrophobic group (R group in general formula (b)) having a hydrophobic interaction.

Item 13. The method according to any one of items 1 to 12, wherein electrical conductivity of the solution containing the antibody and the HCP is 22 mS/cm or less.

Item 14. The method according to any one of items 1 to 13, wherein a polyanion is further contained in the solution containing the antibody and the HCP.

Item 15. The method according to item 14, wherein the polyanion is one or more types selected from the group of a citrate ion, a phosphate ion and a sulfate ion.

Item 16. The method according to any one of items 1 to 15, wherein the antibody is a monoclonal antibody.

### Advantageous Effects of Invention

An embodiment of the invention provides an antibody purification method with a higher purification degree by separating an antibody and impurities from a solution containing the antibody and the impurities such as HCP.

### BRIEF DESCRIPTION OF DRAWINGS

FIG. 1 is a chromatogram obtained by performing size extrusion chromatography in Reference Example 1.

### DESCRIPTION OF EMBODIMENTS

Hereinafter, an embodiment of the invention will be described in detail.

One embodiment of the invention provides an antibody purification method, including bringing a solution containing an antibody and host cell protein (HCP) into contact with a chromatography media to separate the antibody and the HCP, wherein the chromatography media contains a base media containing porous particles, and a compound having a plurality of primary amino groups bonded to the base media, and 20 to 55% of the primary amino groups in the compound having the plurality of primary amino groups are modified with a hydrophobic group.

The chromatography media used in the method according to the embodiment contains the compound having the plurality of primary amino groups as a ligand, and 20 to 55% of the primary amino groups in the compound having the plurality of primary amino groups are modified with the hydrophobic group. When the chromatography media having such a configuration is used in purification of the antibody, a capability of separating the antibody and the impurities (particularly, HCP removability) from the solution containing the antibody and the impurities such as the HCP is high, and the purified antibody can be obtained with a high purification degree and high collection efficiency. In ion exchange chromatography, if electrical conductivity of the solution containing the antibody to be purified and the impurities (hereinafter, also referred to as an “antibody solution”) is increased, reduction of adsorbability and separation ability has so far remained as a problem. Moreover, particularly in anion exchange chromatography utilized in a flow-through mode in antibody pharmaceutical purification, a similar problem has remained also when a polyanion such as a citrate ion, a phosphate ion and a sulfate ion exists in the antibody solution by being contained in a medium or a buffer solution. However, according to the method related to the embodiment, when the electrical conductivity of the antibody solution is comparatively high and/or when the polyanion exists in the antibody solution, high adsorbability and high separation ability can be maintained, and the purified antibodies can be obtained with the high purification degree and the high collection efficiency.

Thus, the method according to the embodiment develops the excellent adsorbability and separation ability regardless of the electrical conductivity of the antibody solution, and therefore can be reasonably used for purification of the antibody solutions in a wide range. Moreover, the antibody solution having the electrical conductivity (about 14 mS/cm) equivalent to the electrical conductivity of an antibody culture solution can be directly provided for a column, and a step of desalting and diluting the antibody solution to adjust the electrical conductivity before purification, which has been performed so far, does not need to be performed, and therefore purification of the antibody can be performed further simply. Further, the polyanion does not need to be removed from the antibody solution before purification, either, and therefore purification of the antibody can be reasonably performed further simply also in view of the above regard.

### 1. Chromatography Media

Hereinafter, each component of the chromatography media used in the embodiment will be described in the order.

### (1) Base Media

The chromatography media generally has a configuration in which a ligand is bonded to a base media. The base media contains porous particles, and the porous particles are modified with a functional group (for example, a hydroxy group or a carbamoyl group) for introducing the compound having the plurality of primary amino groups as the ligand thereinto. The porous particles to be used are not limited as long as the particles can be modified with such a functional group. Specific examples thereof preferably include a polysaccharide such as agarose, dextran, starch, cellulose, pullulan, chitin, chitosan, triacetyl cellulose and diacetyl cellulose, and a derivative thereof; and an organic polymer such as polyacrylamide, polymethacrylamide, polyacrylate, polymethacrylate, polyalkylvinyl ether and polyvinyl alcohol. The porous particles preferably form a crosslinked structure in view of a capability of ensuring mechanical strength. Among the above porous particles, crosslinked cellulose particles in which a skeleton of the cellulose particles is reinforced by a crosslinking reaction is further preferably used.

The crosslinked cellulose particles are not particularly limited as long as the particles can be used as the base media of the chromatography media. The cellulose serving as a raw material may be crystalline cellulose or non-crystalline cellulose, but crystalline cellulose is preferred in view of high strength.

Specific examples of the crosslinked cellulose particles that can be preferably used include porous cellulose gel disclosed in JP 2009-242770 A. The porous cellulose gel disclosed in the same gazette is obtained by a method including a step of adding dropwise continuously or dividedly, to a suspension of uncrosslinked cellulose particles, a crosslinking agent in an amount 4 to 12 times the number of moles of a cellulose monomer, and alkali in an amount 0.1 to 1.5 times the number of moles of the crosslinking agent in 3 hours or more in the presence of at least one kind of inorganic salt selected from the group of hydrochloride, sulfate, phosphate and borate in an amount 6 to 20 times the number of moles of the cellulose monomer.

The thus obtained crosslinked cellulose particles have high mechanical strength and can be used under chromatography conditions in which a flow rate is high, and thus can give a cation exchange chromatography media having high productivity. Here, a term “cellulose monomer” means a glucose unit being a constitutional unit of cellulose. Moreover, the number of moles of the cellulose monomer (namely, a degree of polymerization) is calculated based on an amount obtained by subtracting moisture from one unit of glucose (namely, dry weight of cellulose) (the molecular weight of 162 is taken as 1 mol).

A shape of the porous particles is not particularly limited, and is preferably spherical in view of high mechanical strength, excellent gel precipitability and a capability of preparation of a uniform packed bed. In the above case, sphericity of the porous particles is preferably 0.8 to 1.0. Here, a term “sphericity” means a ratio of a minor axis to a major axis of the porous particles.

Spherical cellulose particles can be easily obtained by dissolving crystalline cellulose or cellulose formed of a crystalline region and a non-crystalline region into a solvent to regenerate the cellulose, for example. Specific examples of a method for producing the spherical cellulose particles include a method through acetate as described in JP S55-39565 B and JP S55-40618 B; a method for producing the cellulose from a solution containing calcium thiocyanate as described in JP S63-62252 B; a method for producing the cellulose from a solution containing paraformaldehyde and dimethylsulfoxide as described in JP S59-38203 A; and a method for producing the cellulose from a cellulose solution in which the cellulose is dissolved into lithium chloride-containing amide as described in JP 3663666 B. Moreover, spherical crosslinked cellulose particles can be obtained by crosslinking the spherical cellulose particles.

A particle size of the porous particles is preferably 10 to 500 micrometers, further preferably 30 to 200 micrometers, and particularly preferably 50 to 150 micrometers. Moreover, a mean particle size is preferably 30 to 1,000 micrometers, further preferably 40 to 200 micrometers, and particularly preferably 50 to 100 micrometers. Here, a term “particle size” means a measured value of the particle size of each porous particle, and a term “mean particle size” means a mean value calculated based on the particle size.

The particle size and the mean particle size of the porous particles herein can be measured using a laser diffraction/scattering particle size distribution measuring device. In the above device, a group of particles is irradiated with a laser beam to determine a particle size distribution from a diffracted or scattered light intensity distribution pattern emitted from the group of particles, and the particle size and the mean particle size are calculated based thereon. As a specific measuring device, Laser Diffraction/Scattering Particle Size Distribution Analyzer LA-950 (made by HORIBA, Ltd.) or the like can be used.

Alternatively, the particle size can also be measured using an image photographed with an optical microscope. Specifically, the particle size on the image is measured using a caliper or the like, and an original particle size is determined using photographing magnification. Then, from a value of each particle size determined from the image of the optical microscope, the mean particle size is calculated using the following formula:

{volume mean particle size(MV)}=Σ(nd⁴)/Σ(nd³)

where, d represents a value of each particle size determined from an image of an optical microscope, and n represents the number of measured particles.

Porosity of the porous particles can be characterized by pore size characteristics. One of indicaters indicating the pore size characteristics includes the gel partition coefficient Kay. The pore size influences physical strength of the particles or diffusivity of an objective material serving as a purification object in the porous particles. Accordingly, a difference is produced in a flow rate of a liquid passing through an inside of the porous particles and dynamic adsorption capacity of the porous particles depending on the pore size. Therefore, a design of the porous particles to be the pore size according to an intended purpose is required. In particular, from a viewpoint of the dynamic adsorption capacity, the gel partition coefficient Kav of the porous particles is preferably in the range of 0.15 to 0.6, further preferably in the range of 0.2 to 0.55 and particularly preferably in the range of 0.3 to 0.5, when standard polyethylene oxide having weight average molecular weight of 1.5×10⁵ Da is used as a sample and pure water is used as a mobile phase.

In the invention according to the embodiment, the porous particles having such a pore size that the gel partition coefficient in the range described above can be obtained are preferably used from a viewpoint of adsorption characteristics. When the crosslinked cellulose particles are used as the porous particles, the gel partition coefficient Kav thereof can be adjusted by controlling a concentration of dissolved cellulose during formation of the particles, for example.

The gel partition coefficient Kav can be determined from a relationship between retention volume and column volume when a standard material (for example, polyethylene oxide) having specific molecular weight was used as a sample, according to the following equation:

Kav=(Ve−V₀)/(Vt−V₀)

where, Ve represents retention volume (mL) of a sample, Vt represents empty column volume (mL), and V₀ represents blue dextran retention volume (mL).

A specific method for measuring the gel partition coefficient Kav is described in Seibutsukagaku Jikkenho (Biochemistry Experimental Method) 2 “Gel chromatography,” first edition, authored by L. Fischer (Tokyo Kagaku Dojin), for example.

### (2) Ligand

The chromatography media used in the method according to the embodiment contains the compound having the plurality of primary amino groups as the ligand, in which 20 to 55% of the primary amino groups in the compound having the plurality of primary amino groups are modified with the hydrophobic group. Here, “the compound having the plurality of primary amino groups” exists in a state in which the compound is bonded to the base media described above, and accordingly a term “compound having the plurality of primary amino groups” herein can also be expressed as “a ligand having the plurality of primary amino groups.”

### Compound Having a Plurality of Primary Amino Groups

First, the compound having the plurality of primary amino groups will be described.

The compound having the plurality of primary amino groups used as the ligand is not particularly limited as long as the compound can be bonded to the functional group on the base media. Specific examples thereof include polyamine such as polyallylamine and polyvinylamine; polysaccharide such as chitosan; and polyamino acid such as polylysine, polyguanidine and polyornithine. Above all, polyallylamine and polylysine are preferred, and polyallylamine is further preferred.

Weight average molecular weight of the compound having the plurality of primary amino groups may be 300,000 or less, preferably 1,000 to 100,000, further preferably 3,000 to 50,000, and particularly preferably 5,000 to 15,000. When the polyallylamine is used, the weight average molecular weight may be 150,000 or less, preferably 1,000 to 100,000, further preferably 3,000 to 50,000, particularly preferably 5,000 to 15,000, and most preferably 10,000 to 15,000.

A method for adding the compound having the plurality of primary amino groups to the base media is not particularly limited, and addition thereof can be performed by a publicly-known method. For example, the addition can be performed, under predetermined conditions, by stirring a solution containing the porous particles modified with the functional group (for example, the hydroxy group, the carbamoyl group or the like) to which the compound having the plurality of primary amino groups can be bonded, and the compound having the plurality of primary amino groups.

Alternatively, the compound having the plurality of primary amino groups may be added thereto by allowing graft polymerization of a monomer on the base media. In the above case, the compound containing the primary amino groups may be used as the monomer, or the compound having the plurality of primary amino groups may be added thereto by allowing graft polymerization of a monomer having a group reactive with amine, such as glycidyl methacrylate, on the base media, and then allowing the resulting material to react with ammonia.

### Hydrophobic Group

The hydrophobic group is not particularly limited as long as the hydrophobic group is bonded to the primary amino group in the compound containing the plurality of primary amino groups and has hydrophobicity, but the hydrophobic group ordinarily used in a hydrophobic chromatography media is preferred. Specific examples of such a hydrophobic group include a group containing a saturated alkyl group and/or a phenyl group. As the saturated alkyl group, a straight-chain saturated alkyl group is preferred, a straight-chain saturated alkyl group having 4 to 8 carbons is further preferred, and an n-butyl group is particularly preferred.

Specific examples of a structure of a preferred hydrophobic group include a structure represented by any one of the following general formulas (1) to (3).

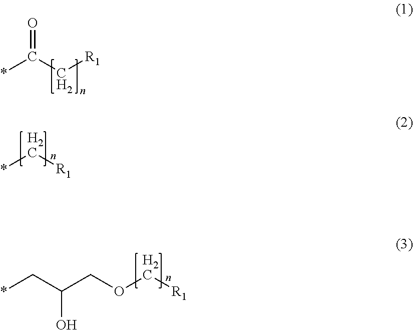

In general formulas (1) to (3),

n is an integer from 0 to 8,

R₁ is a phenyl group when n is an integer from 0 to 3, and H or a phenyl group when n is an integer from 4 to 8, and

an asterisk (*) is a bonding site with the primary amino group in the compound having the plurality of primary amino groups.

In other words, n is an integer from 0 to 8 when R₁ is the phenyl group, and is an integer from 4 to 8 when R₁ is H.

A carbon atom in the structure represented by general formulas (1) to (3) described above may have a substituent such as an alkyl group or an alkoxy group having 1 to 2 carbons, for example, a methyl group, an ethyl group, a methoxy group, an ethoxy group or the like.

Among the structures represented by general formulas (1) to (3) described above, a structure represented by general formula (1) is further preferred. As a further preferred structure, in the structure represented by general formula (1), n is preferably 4.

A bonding mode of the hydrophobic group to the primary amino group is not particularly limited as long as the mode is covalent bonding. Specifically, for example, the mode may be amide bonding formed by a reaction between acid anhydride, acid chloride or active ester and the amino group or carbon-nitrogen bonding formed by a reaction between an epoxy compound or a halide and the amino group.

Specific examples of the compound for introducing the hydrophobic group as described above thereinto include valeric anhydride, caproic anhydride, enanthic anhydride, caprylic anhydride, pelargonic anhydride, benzoic anhydride, butyl glycidyl ether and phenyl glycidyl ether. More specifically, the hydrophobic group is conveniently a group derived from the above compounds. The hydrophobic group can be bonded to the primary amino group of the compound having the plurality of primary amino groups by allowing the above compounds to react with the compound having the plurality of primary amino groups. Among the compounds described above, valeric anhydride and benzoic anhydride are further preferred. Such acid anhydride is preferred in view of efficient progress of the reaction with the compound having the plurality of primary amino groups under mild conditions.

A method upon modifying the primary amino group in the compound having the plurality of primary amino groups with the hydrophobic group is not particularly limited, and a publicly-known method can be used. For example, the modification can be performed by stirring, under predetermined conditions, a solution containing the compound having the plurality of primary amino groups and the compound for introducing the hydrophobic group.

A structure of the hydrophobic group may be represented by the following general formula (4) or (5).

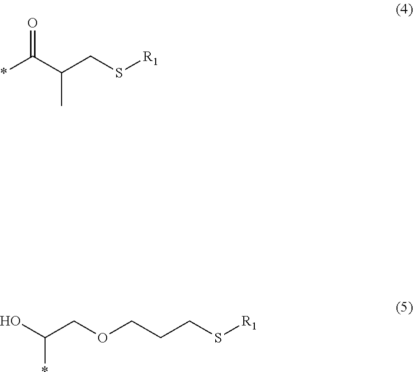

In general formulas (4) and (5), R₁ is a heterocyclic group, and an asterisk (*) represents a bonding site with the primary amino group in the compound having the plurality of primary amino groups.

The heterocyclic group of R₁ is not particularly limited, and is preferably a heterocyclic group containing a nitrogen atom, and further preferably an aromatic heterocyclic group having a nitrogen atom. Specific examples of a heterocycle in the heterocyclic group include pyridine, imidazole, benzimidazole, pyrazole, imidazoline, pyrazine, indole, isoindole, quinoline, isoquinoline and quinoxaline, and pyridine, imidazole and benzimidazole are preferred.

A carbon atom in the heterocyclic group may have a substituent. Specific examples of the substituent include an alkyl group or an alkoxy group having 1 to 4 carbons, and preferably include a methyl group, an ethyl group, a propyl group, a butyl group, a methoxy group, an ethoxy group, a propyloxy group and a butoxy group.

In order to introduce the hydrophobic group represented by general formula (4) described above into the compound having the plurality of primary amino groups, for example, a methacryl group is bonded to the primary amino group in the compound having the plurality of primary amino groups, and further a heterocycle-containing group is bonded to the methacryl group. The methacryl group can be introduced thereinto by allowing the primary amino group in the compound having the plurality of primary amino groups to react with methacrylic anhydride, acid chloride of methacrylic acid, or an active ester compound derived from methacrylic acid, or the like. Moreover, the heterocycle-containing group can be introduced thereinto by allowing the heterocyclic group-containing compound to react with the methacryl group bonded to the primary amino group. The heterocyclic group-containing compound contains the heterocyclic group and a thiol group, for example, and in the above case, the thiol group reacts with the methacryl group.

In order to add the hydrophobic group represented by general formula (5) described above to the compound having the plurality of primary amino groups, an allyl group is bonded to the primary amino group in the compound having the plurality of primary amino groups, and further a heterocycle-containing group is bonded to the allyl group, for example. The allyl group can be introduced thereinto by allowing the primary amino group in the compound having the plurality of primary amino groups to react with a compound (for example, allylglycidylether) simultaneously having the functional group to be bonded to the primary amino group and the allyl group. Moreover, the heterocycle-containing group can be introduced thereinto by allowing the heterocyclic group-containing compound to react with the allyl group bonded to the primary amino group. The heterocyclic group-containing compound contains the heterocyclic group and the thiol group, for example, and in the above case, the thiol group reacts with the allyl group or the functional group derived from the allyl group.

The heterocyclic group-containing compound used in introducing the hydrophobic group represented by general formulas (4) and (5) described above is not particularly limited as long as the heterocyclic group can be introduced thereinto, but the compound containing the heterocyclic group and the thiol group is preferred. Specific examples of such a compound include 2-mercaptoethylpyridine, 2-mercaptobenzimidazole, 2-mercapto-4-methylimidazole and 2-mercapto-4,5-methylimidazole.

In the compound having the plurality of primary amino groups, 20 to 55% of the primary amino groups in the compound are modified with the hydrophobic group. The rate of modification of the amino group is expressed in terms of a value based on the total number of the primary amino groups existing in the compound having the plurality of primary amino groups. For example, when 100 primary amino groups exist in the compound having the plurality of primary amino groups, the values of 20 to 55% mean that 20 to 55 primary amino groups are modified with the hydrophobic group. The rate of modification of the amino group is further preferably 25 to 55%. Alternatively, in one embodiment of the invention, the rate of modification may be 10 to 75%. In addition, when the hydrophobic group includes the saturated alkyl group such as a butyl group and does not include the phenyl group, and when the rate of modification of the amino group is more than 40% to 55%, and further preferably 45% to 55% or 50% to 55%, the amount of the HCP in a collected fraction is small, and the antibody having high purity can be obtained. Moreover, when the hydrophobic group includes the phenyl group, as the rate of modification of the amino group is increased, the collection efficiency of the antibody is recognized to be decreased, and therefore a balance between the collection efficiency of the antibody and the purity is preferably optimized.

Upon allowing the compound having the plurality of primary amino groups to react with the compound for introducing the hydrophobic group, the rate of modification of the amino group can be adjusted to be within the range described above by adjusting an amount of the compound for introducing the hydrophobic groups. The rate of modification of the amino group can be calculated by measuring ion exchange capacity of the chromatography media before and after introducing the hydrophobic group, and comparing the values.

The ligand described above includes a repeating unit represented by the following general formula (a) and a repeating unit represented by the following general formula (b), for example.

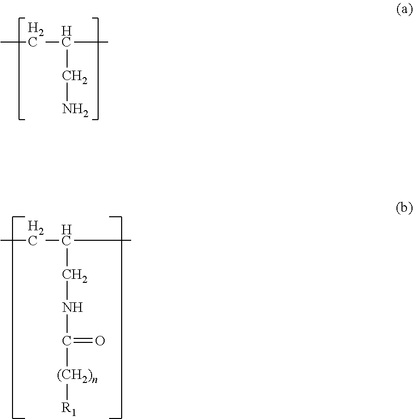

In general formula (b), n and R₁ are as defined in general formula (1) described above. The ligand used in the embodiment preferably has an amino group (—NH₂ group in general formula (a)) being a hydrophilic group and having an electrostatic interaction, an amide group (—NH—CO— group in general formula (b)) having an electrostatic interaction, and a hydrophobic group (—(CH₂)ₙ—R₁ group in general formula (b)) having a hydrophobic interaction, and particularly preferred characteristics are obtained by interaction of three kinds of the groups.

As described above, the method according to the embodiment can be used regardless of the electrical conductivity of the antibody solution, and an antibody solution having comparatively high electrical conductivity of about 22 mS/cm (for example, 14 to 22 mS/cm) can also be purified with high efficiency, for example. Accordingly, according to the method related to the embodiment, the antibody can be purified from an antibody solution having electrical conductivity of 22 mS/cm or less, preferably 2 to 22 mS/cm, and further preferably 6 to 22 mS/cm.

Moreover, as described above, the method according to the embodiment can be used also when the polyanion exists in the antibody solution. Specific examples of the polyanion that can exist in the antibody solution include a citrate ion, a phosphate ion and a sulfate ion, and the polyanion is preferably one or more types selected from the group of a citrate ion, a phosphate ion and a sulfate ion.

### 2. Antibody

Specific examples of the antibody as the purification object include a monoclonal antibody or a polyclonal antibody, and the antibody is preferably a monoclonal antibody. Specific examples of the antibody include a mouse antibody, a lama antibody, a chimeric antibody, a humanized antibody, a human antibody or an antibody obtained by modifying an Fc region thereof, and specific examples of a molecular type include IgG, IgM, IgA, IgD, IgE, Fab, Fc, Fc-fusion protein, VH, VL, VHH, Fab′2, scFv, scFab, scDb and scDbFc.

Moreover, specific examples of the antibody also include a monoclonal antibody or a polyclonal antibody, or partially positively modified monoclonal antibody or polyclonal antibody. Specific example of a method for modifying the monoclonal antibody or the polyclonal antibody include the method described in Journal of PHARMACEUTICAL SCIENCES, 2011, 100, 2104-2119, for example.

The antibody may be a monomer or a polymer, but preferably a monomer. An antibody monomer means a molecule formed of one molecule of antibody. The antibody polymer means a molecule in which two or more molecules of the antibody monomers are polymerized by covalent bonding or noncovalent bonding, and specific examples thereof include a dimer, a trimer, a multimer, an aggregate and an agglomerate.

Specific examples of the impurities contained in the antibody solution include the host cell protein (HCP), and also a material that can be formed in a culture process or any other chromatography treatment step or the like, such as nucleic acid, a virus, a protein A leak, a degradate of the antibody, and a modified antibody subjected to modification, removal of a sugar chain component, oxidation, deamidation or the like.

Specific examples of the antibody solution include a composition obtained from a living body, such as plasma, serum, milk or urine, an antibody-producing cell obtained by using a gene recombination technology or a cell fusion technology, a culture solution of fungi such as Escherichia coli, or a composition obtained from a transgenic nonhuman animal, plant or insect.

Specific examples of the antibody-producing cell include a transformed cell in which a gene encoding a desired antibody is incorporated in a host cell. Specific examples of the host cell include a cell strain such as an animal cell, a plant cell and a yeast cell. Specific examples thereof include a Chinese hamster ovary cell (CHO cell), an NSO cell being a mouse myeloma cell, an SP2/0 cell, a YB2/0 cell being a rat myeloma cell, an IR983F cell, a BHK cell being a Syrian hamster kidney-derived cell, a Namalwa cell being a human myeloma cell, an embryonic stem cell and a fertilized egg cell.

As a medium for culturing the antibody-producing cell, any medium can be used if the medium is suitable for culture of individual cells. Specific examples thereof include a serum-containing medium, a medium containing no animal-derived component such as serum albumin or a serum fraction, a serum-free medium and a protein-free medium, but the serum-free medium or the protein-free medium is preferred. Moreover, when necessary, a physiological active material, a nutrition factor or the like required for growth of the antibody-producing cell can be added thereto. The above additives are previously incorporated into the medium before culture, or appropriately additively supplied to the medium as an additive medium or an additive solution during culture. The additives may be added thereto in one kind or two or more kinds, and the additives may be added thereto continuously or intermittently.

Specific examples of the transgenic nonhuman animal, plant or insect include a nonhuman animal, plant or insect in which a gene encoding protein is incorporated into a cell. Specific examples of the nonhuman animal include a mouse, a rat, a guinea pig, a hamster, a rabbit, a dog, sheep, a pig, a goat, a cow and a monkey. Specific examples of the plant include tobacco, a potato, a tomato, a carrot, a soybean, a rapeseed, alfalfa, rice, wheat, barley and corn.

Moreover, the antibody solution loaded on the chromatography media also includes a solution obtained from a living body, such as plasma and urine containing the antibody as described above, and also an antibody solution obtained in a process of purification. Specific examples of the antibody solution include a cell removal liquid, a precipitate removal liquid, an alcoholic fractionation liquid, a salting-out fractionation liquid and a chromatography effluent. Further, when an insoluble matter such as particles exist in the antibody solution, the insoluble matter may be previously removed, and then the antibody solution may be provided for the purification method according to the embodiment. Specific examples of a method for removing the insoluble matter such as particles include a centrifuge method, a cross flow filtration method (tangential flow filtration method), a filtration method by a depth filter, a filtration method by a membrane filter, a dialysis method and a method in combination of the above methods.

Moreover, when necessary, pH, electrical conductivity, a buffer solution, a salt concentration, an additive and an antibody concentration of the antibody solution, a loaded antibody amount per unit volume of the chromatography media and the like may be previously adjusted to preferable conditions, and then the antibody solution may be provided for the purification method according to the embodiment. Specific examples of the method for adjusting the above values include an ultrafiltration method using an ultrafiltration membrane.

### 3. Purification Method

The antibody purification method according to the embodiment includes bringing the solution containing the antibody and the impurities such as the HCP into contact with the chromatography media described above to separate the antibody and the impurities. Specifically, the antibody can be purified by packing the chromatography media described above into the column, and flowing the antibody solution therethrough, and selectively adsorbing either the antibody or the impurities. Alternatively, the antibody can also be purified by utilizing a difference in affinity relative to the chromatography media by adsorbing both the antibody and the impurities to the media, and stepwise or continuously increasing the salt concentration during elution.

The chromatography media used in the method according to the embodiment has a high capability of adsorbing and separating the impurities such as the HCP contained in the antibody solution, and therefore the flow-through mode is preferably adopted in the method according to the embodiment. Here, the flow-through mode refers to a purification method in which the impurities are bonded to the chromatography media, and an object is flown and collected without being bonded to the chromatography media. For example, when the object is the antibody and the impurities are the host cell protein (HCP), the HCP is bonded to the chromatography media, and the antibody is flown through the column without being bonded to the chromatography media. On the occasion, if the amount is slight, the antibody may be bonded thereto, but the HCP is bonded further selectively to the chromatography media, and thus the antibody is purified.

In contrast, a bind-and-elute mode refers to a purification method in which an object is once bonded to the chromatography media, and then the object is eluted and collected. For example, when the antibody is purified, first, the antibody is bonded to the chromatography media, and the impurities are passed through the column without being bonded to the chromatography media. Then, only the antibody is eluted to the mobile phase by using the mobile phase having a suitable salt concentration or pH, and collected. Specific examples of an elution method include a one-step elution method in which the antibody is eluted by being passed through a buffer solution having a specific salt concentration or pH at which the affinity between the antibody and the chromatography media is decreased, a stepwise method in which the antibody is eluted by stepwise changing a salt concentration or pH value, or a gradient method in which the antibody is eluted by continuously changing a salt concentration or pH.

In setting of chromatography conditions, the difference in the affinity between the antibody and the impurities relative to the chromatography media is utilized. For example, the conditions are set in considering a difference in a media structure (such as ligand species, ligand density, ligand orientation, a particle size, a pore size and a base matrix composition), and physical and chemical properties of the antibody and the impurities (such as an isoelectric point, an electric charge, a hydrophobic degree, a molecular size and a three-dimensional structure).

A component contained in the buffer solution used for the antibody solution and washing of the column or elution is not particularly limited as long as the component has buffer capacity, and specific examples thereof include phosphate, citrate, acetate, succinate, maleate, borate, Tris (base), HEPES, MES, PIPES, MOPS, TES and Tricine, each in an amount of 1 to 300 mmol/L. Moreover, the salt described above can also be used in combination with any other salt such as sodium chloride, potassium chloride, calcium chloride, sodium citrate, sodium sulfate and ammonium sulfate, for example. Further, the buffer solution may contain amino acid such as glycine, alanine, arginine, serine, threonine, glutamic acid, aspartic acid and histidine; sugar such as glucose, sucrose, lactose and sialic acid; or a derivative thereof, for example.

The pH of the buffer solution used for the antibody solution and washing of the column or elution is preferably in the range of 2 to 9, and further preferably in the range of 3 to 8.

A linear velocity of the buffer solution used for the antibody solution and washing of the column or elution is preferably in the range of 50 to 1,000 cm/h.

The loaded antibody amount per unit volume of the chromatography media is preferably 10 to 500 g/L, and further preferably 60 to 200 g/L.

The purification method according to the embodiment may be performed in combination with any other purification method. As any other purification method, any method can be used if the method is suitable for purification of the antibody, and specific examples thereof include chromatography, activated carbon treatment, alcoholic fractionation, precipitate removal, salting-out, buffer solution exchange, concentration, dilution, filtration, virus inactivation and virus removal. As any other purification method, one or more methods may be selected, and may be performed before or after the purification method according to the embodiment.

Moreover, a cation exchanger and an anion exchanger are generally used in the bind-and-elute mode and in the flow-through mode, respectively, in a production field of the monoclonal antibody, but if the chromatography media of the invention is used, the chromatography media can also be adapted for a method in which the cation exchanger is used in the flow-through mode.

When any other purification method is chromatography, specific examples of the media or the membrane to be used include an affinity media such as a heparin media and a protein A media, a cation exchange media, a cation exchange membrane, an anion exchange media, an anion exchange membrane, a gel filtration media, a hydrophobic interaction media, a reversed phase media, a hydroxyapatite media, a fluoroapatite media, a sulfated cellulose media, a sulfated agarose media and a mixed-mode (multimodal) media.

According to the method related to the embodiment, the antibody can be purified at the collection efficiency of 85% or more, and preferably 90% or more. Here, the collection efficiency means a proportion of an amount of the antibody collected therein to an amount of the antibody loaded on the chromatography media (namely, an amount of the antibody in the antibody solution before purification). Moreover, as described above, the chromatography media to be used in the method according to the embodiment is excellent in the capability of adsorbing the impurities such as the HCP in the antibody solution, and therefore the antibody can be obtained with a high purification degree. Specifically, the amount of the HCP contained in the antibody solution (collected fraction) after purification is preferably less than 50 ppm (0 to less than 50 ppm), further preferably 35 ppm or less (0 to 35 ppm), still further preferably 25 ppm or less (0 to 25 ppm), and particularly preferably 10 ppm or less (0 to 10 ppm). In addition, the amount of the HCP described above is expressed in terms of a value calculated from the following equation: {amount of HCP in antibody solution after purification (ng)}/{amount of antibody in antibody solution after purification (mg)}.

The preferred amount of the HCP described above can be achieved regardless of the electrical conductivity levels of the antibody solution, and in both cases where the electrical conductivity of the antibody solution before purification is 6 mS/cm and 14 mS/cm, the amount of the HCP contained in the antibody solution (collected fraction) after purification is preferably less than 45 ppm (0 to less than 45 ppm), further preferably 35 ppm or less (0 to 35 ppm), still further preferably 25 ppm or less (0 to 25 ppm), and particularly preferably 10 ppm or less (0 to 10 ppm). In particular, the antibody solution is provided for cation chromatography, and then the purification method according to the embodiment is performed, and thus even the antibody solution having comparatively high electrical conductivity can be purified at a high purification degree (namely, a low amount of the HCP). Moreover, according to the preferred aspect of the invention, even when the polyanion exists in the antibody solution, the antibody can be purified at the collection efficiency and in the amount of the HCP as described above.

### EXAMPLES

### 1. Production of a Chromatography Media

### (1) Media a (No Hydrophobic Group)

### Production of 6% Spherical Cellulose Particles (Hydrous)

Then, 6% spherical cellulose particles were produced according to procedures described below. Here, when a concentration of crystalline cellulose in a step of (i) described below is 6% by weight, cellulose particles to be produced is referred to as “6% spherical cellulose particles.”

(i) To 100 g of 60 wt % calcium thiocyanate aqueous solution, 6.4 g of crystalline cellulose (made by Asahi Kasei Chemicals Corporation, trade name: CEOLUS PH101) was added, and the resulting mixture was heated to 110 to 120° C. and dissolved.

(ii) Then, 6 g of sorbitan monooleate was added to the above solution as a surfactant. The resulting mixture was added dropwise to 480 mL of o-dichlorobenzene preheated to 130 to 140° C., and the resulting mixture was stirred at 200 to 300 rpm to obtain a dispersion.

(iii) Then, the dispersion was cooled to 40° C. or lower. The resulting solution was poured into 190 mL of methanol to obtain a suspension of particles.

(iv) The suspension obtained was subjected to filtration and fractionated to collect particles, and the particles were washed with 190 mL of methanol. The above washing operation was performed several times.

(v) The particles were further washed with a large amount of water to obtain spherical cellulose particles.

(vi) Then, the spherical cellulose particles were sieved through a sieve having 53 μm to 125 μm according to a JIS standard sieve specification to obtain 6% spherical cellulose particles having a desired particle size (particle size: 50 to 150 μm, mean particle size: about 100 μm) (hydrous, cellulose dissolved concentration: 6% by weight).

In addition, the mean particle size herein was measured using an image photographed by an optical microscope. Specifically, the particle size on the image was measured using a caliper to determine an original particle size from photographing magnification. Then, the mean particle size was calculated from values of individual particle size values determined from the image of the optical microscope, according to the following equation:

{volume mean particle size(MV)}=Σ(nd⁴)/Σ(nd³)

where, d represents a value of each particle size determined from an image of an optical microscope, and n represents the number of measured particles.

### Production of Crosslinked 6% Cellulose Particles

Then, crosslinked 6% cellulose particles were produced by allowing a crosslinking reaction of the 6% spherical cellulose particles produced as described above. The procedures are as described below.

(i) To 100 g of the 6% spherical cellulose particles (hydrous) obtained as described above, 121 g of pure water was added, and the resulting mixture was warmed while the mixture was stirred. When temperature reached 30° C., 3.3 g of a 45 wt % NaOH aqueous solution and 40.5 g of NaBH were added thereto, and the resulting mixture was further warmed and stirred. An initial alkali concentration herein was 0.69% (w/w).

(ii) After 30 minutes, 60 g of Na₂SO₄ was added to the reaction mixture and dissolved therein. At a time point at which the temperature reached 50° C., stirring was further continued for 2 hours while the temperature was maintained at 50° C.

(iii) While stirring of the resulting mixture was continued at 50° C., an amount obtained by equally dividing into 25 each of 48 g of 45 wt % NaOH aqueous solution and 50 g of epichlorohydrin was added thereto every 15 minutes in about 6 hours.

(iv) After completion of addition, the resulting mixture was allowed to react at a temperature of 50° C. for 16 hours.

(v) After the resulting reaction mixture was cooled to 40° C. or lower, 2.6 g of acetic acid was added thereto to neutralize the resulting mixture.

(vi) The resulting reaction mixture was subjected to filtration to collect cellulose particles, and the cellulose particles were subjected to filtration and washed with pure water to obtain crosslinked 6% cellulose particles.

A mean particle size and a Kav value of the crosslinked 6% cellulose particles obtained were measured as described below.

### Measurement of a Mean Particle Size

When the mean particle size was measured using Laser Diffraction/Scattering Particle Size Distribution Analyzer LA-950 (made by HORIBA, Ltd.), the mean particle size was 85 μm.

### Measurement of a Kay Value

The gel partition coefficient Kav was calculated from a relationship between retention volume and column volume thereof by using standard polyethylene oxide having weight average molecular weight of 1.5×10⁵ Da as a sample, according to the following equation. In addition, pure water was used as a mobile phase.

Kav=(Ve−V₀)/(Vt−V₀)

where, Ve represents retention volume (mL) of a sample, Vt represents empty column volume (mL), and V₀ represents blue dextran retention volume (mL).

The gel partition coefficient Kav of the crosslinked 6% cellulose particles obtained as described above was 0.38.

### Epoxidation of Crosslinked 6% Cellulose Particles

To a 10 L SUS vessel, 3,000 g of the wet cellulose particles obtained as described above and 1952 g of pure water were added, and the resulting mixture was takes as slurry. Then, 1764 g of epichlorohydrin was added thereto. A temperature of the resulting mixture was increased up to 28° C., and a 48.7% sodium hydroxide aqueous solution was added dropwise thereonto for 2 hours at a liquid temperature no more than 30° C. After completion of dropwise addition, the resulting mixture was stirred at 30° C. for 3 hours. Then, 145 g of acetic acid was added thereto, and the resulting mixture was stirred for 10 minutes. After completion of reaction, wet particles were subjected to filtration, and the wet particles collected were washed with 6 L of pure water 16 times to obtain objective epoxidized cellulose particles.

### Addition of Polyallylamine

In a 10 L SUS vessel, 3,000 g of the epoxidized cellulose particles obtained as described above and 4995 g of PA-15C (made by Nittobo Medical Co., Ltd.) being a 15.3% aqueous solution of polyallylamine having weight average molecular weight of 15,000 was put, and the resulting mixture was stirred at 45° C. for 18 hours. After completion of reaction, wet particles were subjected to filtration and the wet particles collected were washed with 6 L of pure water 10 times to obtain objective polyallylamine-added cellulose particles (media A). Ion exchange capacity of the polyallylamine-added cellulose particles was 0.23 mmol/mL. A method for measuring the ion exchange capacity is as described later.

### (2) Media B (Hydrophobic Group-Added; Valeric Anhydride; Rate of Modification of an Amino Group 26%)

Then, 30 g of media A obtained as described above was washed with 90 mL of methanol5 times. The particles washed with methanol and 50 mL of methanol were added to a 150 mL vessel, and the resulting mixture was taken as slurry. Then, 0.57 g of valeric anhydride and 0.31 g of triethylamine were added thereto, and the resulting mixture was stirred at 25° C. for 24 hours. After completion of reaction, wet particles were subjected to filtration and the wet particles collected were washed with 45 mL of methanol once, with 45 mL of a 0.1 M sodium hydroxide aqueous solution once, and with 45 mL of pure water 10 times to obtain an object. Ion exchange capacity of the particles obtained was 0.17 mmol/mL.

### (3) Media C (Hydrophobic Group-Added; Valeric Anhydride; Rate of Modification of an Amino Group 52%)

Media C was produced in a manner similar to the method in media B except that an amount of valeric anhydride and an amount of triethylamine were changed to 1.12 g and 0.61 g, respectively. Ion exchange capacity of the particles obtained was 0.11 mmol/mL.

### (4) Media D (Hydrophobic Group-Added; Benzoic Anhydride; Rate of Modification of an Amino Group 26%)

Media D was produced in a manner similar to the method in media B except that 0.57 g of benzoic anhydride was used in place of valeric anhydride. Ion exchange capacity of the particles obtained was 0.17 mmol/mL.

### (5) Media E (Hydrophobic Group-Added; Benzoic Anhydride; Rate of Modification of an Amino Group 52%)

Media E was produced in a manner similar to the method in media B except that 1.36 g of benzoic anhydride was used in place of valeric anhydride, and an amount of triethylamine was changed to 0.60 g. Ion exchange capacity of the particles obtained was 0.11 mmol/mL.

### 2. Preparation of an Antibody Solution

### (1) Antibody Solution a

### Purification by a Protein a Column

(i) Use resin and system

Protein A resin: KANEKA KanCap A (KANEKA CORPORATION)

Column: inner diameter 2.6 cm, height 40 cm

System: Akta avant 25

(ii) Culture solution and solutions used for purification

Culture solution: CHO cell culture solution in which a monoclonal antibody (IgG1) was produced (decelluralized)

A1 buffer: 20 mM sodium phosphate buffer solution (pH 7.4)+0.15 M NaCl

A2 buffer: 20 mM sodium phosphate buffer solution (pH 7.4)

B1 buffer: 60 mM sodium citrate buffer solution (pH 3.5)

0.1 M sodium hydroxide aqueous solution

(iii) Procedures

The protein A resin was packed into in the column up to a height of 10 cm. The column was connected to the system, and the A1 buffer by a volume of 2 columns was passed through the column at 13.25 mL/min to equilibrate the column. All subsequent steps were also performed at a flow rate of 13.25 mL/min. Then, 1400 mL of the culture solution was passed through the column. An adsorbed culture solution was washed with the A1 buffer by a volume of 3 columns, and then the A2 buffer by a volume of 2 columns was further passed through the column. Then, the B1 buffer by a volume of 4.8 columns was passed through the column to elute the monoclonal antibody adsorbed to the protein A resin. Collection of the antibody was confirmed by measuring absorbance at a measuring wavelength of 280 nm, and a volume of about 2 columns in a volume of 4.8 columns was collected as a collected fraction. Then, the A1 buffer by a volume of 2 columns and the 0.1 M sodium hydroxide aqueous solution by a volume of 3 columns were passed through the column after elution to wash the column. Finally, the A1 buffer by a volume of 5 columns was passed through the column to reequilibrate the column.

### Virus Inactivation Treatment of a Collected Fraction

Then, 0.1 M of citric acid was added to the collected fraction obtained as described above until pH reached 3.4. After the resulting mixture was left to stand at 25° C. for 1 hour, a 1 M trishydroxyaminomethane aqueous solution was added thereto until pH reached 7.0. Turbidity was confirmed, and therefore the resulting mixture was filtrated with filters having pore sizes of 1.2 μm and 0.45 μm, respectively. A concentration of a monoclonal antibody in a filtrate was 16.37 mg/mL, and an amount of HCP was 184 ppm.

### Solution Preparation

A part of the filtrate obtained as described above was diluted with ultra pure water. Then, the resulting mixture was adjusted to pH of 7.0 and electrical conductivity of 6 mS/cm by using a 1 M trishydroxyaminomethane aqueous solution and a 5M sodium chloride aqueous solution to obtain antibody solution a. A concentration of a monoclonal antibody in antibody solution a was 10.42 mg/mL.

### (2) Antibody Solution b

Antibody solution b was prepared in a manner similar to the method in antibody solution a except that the electrical conductivity was adjusted to 14 mS/cm in the step of solution preparation. A concentration of a monoclonal antibody in antibody solution b was 10.63 mg/mL.

### (3) Antibody Solution c

### Purification by Protein a Column

### (i) Use Resin and System

The same as in antibody solution a

### (ii) Culture Solution and Solutions Used for Purification

Culture solution: CHO cell culture solution in which a monoclonal antibody (IgG1) was produced (decelluralized)

A1 buffer: 20 mM sodium phosphate buffer solution (pH 7.4)+0.15 M NaCl

A2 buffer: 20 mM sodium phosphate buffer solution (pH 7.4)

B2 buffer: 60 mM sodium acetate buffer solution (pH 3.5)

0.1 M sodium hydroxide aqueous solution

(iii) Procedures

Treatment was performed in a manner similar to the treatment in antibody solution a except that the B2 buffer was used in place of the B1 buffer.

### Virus Inactivation Treatment of a Collected Fraction

Then, 1 M hydrochloric acid was added to the collected fraction obtained as described above until pH reached 3.4. The resulting mixture was left to stand at 25° C. for 1 hour, and then the 1 M sodium hydroxide aqueous solution was added thereto until pH reached 5.0. Turbidity was confirmed, and therefore the resulting mixture was filtrated with filters having pore sizes of 1.2 μm and 0.45 μm, respectively. A concentration of a monoclonal antibody in a filtrate was 18.17 mg/mL, and an amount of HCP was 72 ppm.

### Solution Preparation

A part of the filtrate obtained as described above was diluted with ultra pure water. Then, the resulting mixture was adjusted to pH of 7.0 and electrical conductivity of 6 mS/cm by using a 1 M trishydroxyaminomethane aqueous solution and a 5 M sodium chloride aqueous solution to obtain antibody solution c. A concentration of a monoclonal antibody in antibody solution c was 10.79 mg/mL.

### (4) Antibody Solution d

Antibody solution d was prepared in a manner similar to the method in antibody solution c except that the electrical conductivity was adjusted to 14 mS/cm in the step of solution preparation. A concentration of a monoclonal antibody in antibody solution d was 10.48 mg/mL.

### (5) Antibody Solution e

### Purification by a Protein a Column

### (i) Use Resin and System

The same as in antibody solution a

### (ii) Culture Solution and Solutions Used for Purification

Culture solution: CHO cell culture solution in which a monoclonal antibody (IgG1) was produced (decelluralized)

A3 buffer: 20 mM Tris-HCl buffer solution (pH 7.4)+0.15 M NaCl

A4 buffer: 20 mM Tris-HCl buffer solution (pH 7.4)

B2 buffer: 60 mM sodium acetate buffer solution (pH 3.5)

0.1 M sodium hydroxide aqueous solution

(iii) Procedures

The protein A resin was packed into the column up to a height of 10 cm. The column was connected to the system, and the A3 buffer by a volume of 2 columns was passed through the column at 13.25 mL/min to equilibrate the column. All subsequent steps were also performed at a flow rate of 13.25 mL/min. Then, 1500 mL of the culture solution was passed through the column. An adsorbed culture solution was washed with the A3 buffer by a volume of 3 columns, and then the A4 buffer by a volume of 2 columns was further passed through the column. Then, the B2 buffer by a volume of 4.8 columns was passed through the column to elute the monoclonal antibody adsorbed to the protein A resin. Collection of the antibody was confirmed by measuring absorbance at a measuring wavelength of 280 nm, and a volume of about 2 columns in a volume of 4.8 columns was collected as a collected fraction. Then, the A3 buffer by a volume of 2 columns and the 0.1 M sodium hydroxide aqueous solution by a volume of 3 columns were passed through the column after elution to wash the column. Finally, the A3 buffer by a volume of 5 columns was passed through the column to reequilibrate the column.

### Virus Inactivation Treatment of a Collected Fraction

Then, 1 M hydrochloric acid was added to the collected fraction obtained as described above until pH reached 3.4. The resulting mixture was left to stand at 25° C. for 1 hour, and then the 1 M sodium hydroxide aqueous solution was added thereto until pH reached 5.0. Turbidity was confirmed, and therefore the resulting mixture was filtrated with filters having pore sizes of 1.2 μm, 0.45 μm and 0.2 μm, respectively. A concentration of a monoclonal antibody in a filtrate was 16.5 mg/mL, and an amount of HCP was 955 ppm.

### Solution Preparation

A part of the filtrate obtained as described above was adjusted to pH of 7.0 and electrical conductivity of 22 mS/cm by using a 1 M trishydroxyaminomethane aqueous solution and a 5 M sodium chloride aqueous solution to obtain antibody solution e. A concentration of a monoclonal antibody in antibody solution e was 14.00 mg/mL.

### (6) Antibody Solution f

Then, γ-Globulin, from Human Serum (Wako Pure Chemical Corporation) as a reagent was dissolved in a 20 mM sodium phosphate buffer solution (pH 6.5) to obtain antibody solution f. A concentration of antibody solution f was 9.43 mg/mL. Purity of a monomer as measured with size exclusion chromatography was 84.5%.

### (7) Antibody Solution g

### Purification by a Protein a Column

### (i) Use Resin and System

The same as in antibody solution a.

### (ii) Culture Solution and Solutions Used for Purification

Culture solution: CHO cell culture solution in which a monoclonal antibody (IgG1) was produced (decelluralized)

A3 buffer: 20 mM Tris-HCl buffer solution (pH 7.4)+0.15 M NaCl

A4 buffer: 20 mM Tris-HCl buffer solution (pH 7.4)

B2 buffer: 60 mM sodium acetate buffer solution (pH 3.5)

0.10 M sodium hydroxide aqueous solution

(iii) Procedures

The protein A resin was packed into the column up to a height of 10 cm. The column was connected to the system, and the A3 buffer by a volume of 2 columns was passed through the column at 13.25 mL/min to equilibrate the column. All subsequent steps were also performed at a flow rate of 13.25 mL/min. Then, 800 mL of the culture solution was passed through the column. An adsorbed culture solution was washed with the A3 buffer by a volume of 3 columns, and then the A4 buffer by a volume of 2 columns was further passed through the column. Then, the B2 buffer by a volume of 4.8 columns was passed through the column to elute a monoclonal antibody adsorbed to the protein A resin. Collection of the antibody was confirmed by measuring absorbance at a measuring wavelength of 280 nm, and about a volume of about 2 columns in a volume of 4.8 columns was collected as a collected fraction. Then, the A3 buffer by a volume of 2 columns and the 0.10 M sodium hydroxide aqueous solution by a volume of 3 columns were passed through the column after elution to wash the column. Finally, the A3 buffer by a volume of 5 columns was passed through the column to reequilibrate the column.

### Virus Inactivation Treatment of a Collected Fraction

Then, 1 M hydrochloric acid was added to the collected fraction obtained as described above until pH reached 3.4. The resulting mixture was left to stand at 25° C. for 1 hour, and then the 1 M sodium hydroxide aqueous solution was added thereto until pH reached 5.0. Turbidity was confirmed, and therefore the resulting mixture was filtrated with a filter. A concentration of a monoclonal antibody in a filtrate was 13.10 mg/mL, and an amount of HCP was 480 ppm.

### Solution Preparation

A part of the filtrate obtained as described above was adjusted to electrical conductivity of 22 mS/cm by using a 5 M sodium chloride aqueous solution to obtain antibody solution g. A concentration of a monoclonal antibody in antibody solution g was 12.9 mg/mL.

### (8) Antibody Solution h

### Cation Exchange Chromatography Step

### (i) Chromatography Media and System

Media: Cellufine MAX GS

Column: inner diameter 0.5 cm, height 2.5 cm

System: Akta avant 25

### (ii) Antibody Solution and Solutions Used for Purification

Antibody solution: antibody solution g

A6 buffer: 20 mM Acetate-Na buffer solution+NaCl (pH 5.0, 22 mS/cm)

1 M NaCl aqueous solution

1 M sodium hydroxide aqueous solution

(iii) Procedures

The media was packed into the column up to a height of 2.5 cm. The column was connected to the system, and the A6 buffer by a volume of 5 columns was passed through the column at 0.245 mL/min to equilibrate the column. Then, 16 mL of the antibody solution was passed through the column at 0.245 mL/min. Then, the A6 buffer by a volume of 10 columns was passed through the column at 0.245 mL/min to wash the column. Then, the 1 M NaCl aqueous solution by a volume of 10 columns was passed through the column at 0.245 mL/min. Then, the 1.0 M sodium hydroxide aqueous solution by a volume of 5 columns was passed through the column at 0.245 mL/min to wash the column. Finally, the A6 buffer by a volume of 10 columns was passed through the column at 1.0 mL/min to reequilibrate the column.

A total amount of 16 mL of liquid passed through the column during passing the antibody solution therethrough, and 2.5 mL of a column washing liquid during washing of unadsorbed material were combined, and the resulting mixture was taken as a collected fraction. Collection efficiency of a monoclonal antibody (a proportion of an amount of the antibody collected therein to an amount of the antibody loaded on the chromatography media) was 96%, and an amount of HCP in the collected fraction was 253 ppm.

### Solution Preparation

A part of the collected fraction obtained as described above was adjusted to pH of 7.0 and electrical conductivity of 22 mS/cm by using a 1 M trishydroxyaminomethane aqueous solution and a 5 M sodium chloride aqueous solution to obtain antibody solution h. A concentration of a monoclonal antibody in antibody solution h was 9.41 mg/mL.

### 3. Purification of Antibody

### Example 1

### (i) Chromatography Media and System

Media: media B

Column: inner diameter 0.5 cm, height 3 cm

System: Akta avant 25

### (ii) Antibody Solution and Solutions Used for Purification

Antibody solution: antibody solution a

A3 buffer: 20 mM Tris-HCl buffer solution (pH 7.0)+NaCl (6 mS/cm)

B2 buffer: 20 mM Tris-HCl buffer solution (pH 7.0)+1 M NaCl

0.5 M sodium hydroxide aqueous solution

(iii) Procedures

The media was packed into the column up to a height of 1.5 cm. The column was connected to the system, and the A3 buffer by a volume of 10 columns was passed through the column at 0.3 mL/min to equilibrate the column. Then, 5.4 mL of the antibody solution was passed through the column at 0.075 mL/min. Then, the A3 buffer by a volume of 10 columns was passed through the column at 0.075 mL/min to wash the column. Then, the B2 buffer by a volume of 10 columns was passed through the column at 0.3 mL/min. Then, the 0.5 M sodium hydroxide aqueous solution by a volume of 5 columns was passed through the column at 0.075 mL/min to wash the column. Finally, the A3 buffer by a volume of 20 columns was passed through the column at 0.3 mL/min to reequilibrate the column.

A total amount of 5.4 mL of liquid passed through the column during passing the antibody solution therethrough, and 1.8 mL of a column washing liquid during washing of unadsorbed material were combined, and the resulting mixture was taken as a collected fraction. Collection efficiency of a monoclonal antibody (a proportion of an amount of the antibody collected therein to an amount of the antibody loaded on the chromatography media) was 96%, and an amount of HCP in the collected fraction was 22 ppm.

### Example 2

An antibody was purified in a manner similar to the method in Example 1 except that antibody C was used as the chromatography media. Collection efficiency of a monoclonal antibody was 95% and an amount of HCP in a collected fraction was 23 ppm.

### Example 3

An antibody was purified in a manner similar to the method in Example 1 except that antibody D was used as the chromatography media. Collection efficiency of a monoclonal antibody was 95% and an amount of HCP in a collected fraction was 22 ppm.

### Example 4

An antibody was purified in a manner similar to the method in Example 1 except that antibody E was used as the chromatography media. Collection efficiency of a monoclonal antibody was 90% and an amount of HCP in a collected fraction was 21 ppm.

### Comparative Example 1

An antibody was purified in a manner similar to the method in Example 1 except that Cellufine MAX Q-h (made by JNC Corporation) was used as the chromatography media. Collection efficiency of a monoclonal antibody was 98% and an amount of HCP in a collected fraction was 114 ppm.

### Comparative Example 2

An antibody was purified in a manner similar to the method in Example 1 except that Capto Q (made by GE Healthcare) was used as the chromatography media. Collection efficiency of a monoclonal antibody was 97% and an amount of HCP in a collected fraction was 133 ppm.

### Comparative Example 3

An antibody was purified in a manner similar to the method in Example 1 except that media A was used as the chromatography media. Collection efficiency of a monoclonal antibody was 97% and an amount of HCP in a collected fraction was 145 ppm.

### Example 5

### (i) Chromatography Media and System

Media: media B

Column: inner diameter 0.5 cm, height 3 cm

System: Akta avant 25

### (ii) Antibody Solution and Solutions Used for Purification

Antibody solution: antibody solution b

A4 buffer: 20 mM Tris-HCl buffer solution (pH 7.0)+NaCl (14 mS/cm)

B2 buffer: 20 mM Tris-HCl buffer solution (pH 7.0)+1 M NaCl

0.5 M sodium hydroxide aqueous solution

(iii) Procedures

The media was packed into the column up to a height of 1.5 cm. The column was connected to the system, and the A4 buffer by a volume of 10 columns was passed through the column at 0.3 mL/min to equilibrate the column. Then, 5.4 mL of the antibody solution was passed through the column at 0.075 mL/min. Then, the A4 buffer by a volume of 10 columns was passed through the column at 0.075 mL/min to wash the column. Then, the B2 buffer by a volume of 10 columns was passed through the column at 0.3 mL/min. Then, the 0.5 M sodium hydroxide aqueous solution by a volume of 5 columns was passed through the column at 0.075 mL/min to wash the column. Finally, the A4 buffer by a volume of 20 columns was passed through the column at 0.3 mL/min to reequilibrate the column.

A total amount of 5.4 mL of liquid passed through the column during passing the antibody solution therethrough, and 1.8 mL of a column washing liquid during washing of unadsorbed material were combined, and the resulting mixture was taken as a collected fraction. Collection efficiency of a monoclonal antibody was 97% and an amount of HCP in the collected fraction was 35 ppm.

### Example 6

An antibody was purified in a manner similar to the method in Example 5 except that antibody C was used as the chromatography media. Collection efficiency of a monoclonal antibody was 96% and an amount of HCP in a collected fraction was 22 ppm.

### Example 7

An antibody was purified in a manner similar to the method in Example 5 except that antibody D was used as the chromatography media. Collection efficiency of a monoclonal antibody was 95% and an amount of HCP in a collected fraction was 42 ppm.

### Example 8

An antibody was purified in a manner similar to the method in Example 5 except that antibody E was used as the chromatography media. Collection efficiency of a monoclonal antibody was 91% and an amount of HCP in a collected fraction was 19 ppm.

### Comparative Example 4

An antibody was purified in a manner similar to the method in Example 5 except that Cellufine MAX Q-h (made by JNC Corporation) was used as the chromatography media. Collection efficiency of a monoclonal antibody was 97% and an amount of HCP in a collected fraction was 147 ppm.

### Comparative Example 5

An antibody was purified in a manner similar to the method in Example 5 except that antibody A was used as the chromatography media. Collection efficiency of a monoclonal antibody was 99% and an amount of HCP in a collected fraction was 145 ppm.

### Example 9

### (i) Chromatography Media and System

Media: media C

Column: inner diameter 0.5 cm, height 3 cm

System: Akta avant 25

### (ii) Antibody Solution and Solutions Used for Purification

Antibody solution: antibody solution c

A3 buffer: 20 mM Tris-HCl buffer solution (pH 7.0)+NaCl (6 mS/cm)

B2 buffer: 20 mM Tris-HCl buffer solution (pH 7.0)+1 M NaCl

0.5 M sodium hydroxide aqueous solution

(iii) Procedures

The media was packed into the column up to a height of 1.5 cm. The column was connected to the system, and the A3 buffer by a volume of 10 columns was passed through the column at 0.3 mL/min to equilibrate the column. Then, 5.4 mL of the antibody solution was passed through the column at 0.075 mL/min. Then, the A3 buffer by a volume of 10 columns was passed through the column at 0.075 mL/min to wash the column. Then, the B2 buffer by a volume of 10 columns was passed through the column at 0.3 mL/min. Then, the 0.5 M sodium hydroxide aqueous solution by a volume of 5 columns was passed through the column at 0.075 mL/min to wash the column. Finally, the A3 buffer by a volume of 20 columns was passed through the column at 0.3 mL/min to reequilibrate the column.

A total amount of 5.4 mL of liquid passed through the column during passing the antibody solution therethrough, and 1.8 mL of a column washing liquid during washing of unadsorbed material were combined, and the resulting mixture was taken as a collected fraction. Collection efficiency of a monoclonal antibody was 95% and an amount of HCP in the collected fraction was 3 ppm.

### Comparative Example 6

An antibody was purified in a manner similar to the method in Example 9 except that Cellufine MAX Q-h (made by JNC Corporation) was used as the chromatography media. Collection efficiency of a monoclonal antibody was 97% and an amount of HCP in a collected fraction was 22 ppm.

### Comparative Example 7

An antibody was purified in a manner similar to the method in Example 9 except that Capto Q (made by GE Healthcare) was used as the chromatography media. Collection efficiency of a monoclonal antibody was 96% and an amount of HCP in a collected fraction was 27 ppm.

### Example 10

### (i) Chromatography Media and System

Media: media B

Column: inner diameter 0.5 cm, height 3 cm

System: Akta avant 25

### (ii) Antibody Solution and Solutions Used for Purification

Antibody solution: antibody solution d

A4 buffer: 20 mM Tris-HCl buffer solution (pH 7.0)+NaCl (14 mS/cm)

B2 buffer: 20 mM Tris-HCl buffer solution (pH 7.0)+1 M NaCl

0.5 M sodium hydroxide aqueous solution

(iii) Procedures

The media was packed into the column up to a height of 1.5 cm. The column was connected to the system, and the A4 buffer by a volume of 10 columns was passed through the column at 0.3 mL/min to equilibrate the column. Then, 5.4 mL of the antibody solution was passed through the column at 0.075 mL/min. Then, the A4 buffer by a volume of 10 columns was passed through the column at 0.075 mL/min to wash the column. Then, the B2 buffer by a volume of 10 columns was passed through the column at 0.3 mL/min. Then, the 0.5 M sodium hydroxide aqueous solution by a volume of 5 columns was passed through the column at 0.075 mL/min to wash the column. Finally, the A3 buffer by a volume of 20 columns was passed through the column at 0.3 mL/min to reequilibrate the column.

A total amount of 5.4 mL of liquid passed through the column during passing the antibody solution therethrough, and 1.8 mL of a column washing liquid during washing of unadsorbed liquid were combined, and the resulting mixture was taken as a collected fraction. Collection efficiency of a monoclonal antibody was 96% and an amount of HCP in the collected fraction was 8 ppm.

### Example 11

An antibody was purified in a manner similar to the method in Example 10 except that antibody C was used as the chromatography media. Collection efficiency of a monoclonal antibody was 94% and an amount of HCP in a collected fraction was 5 ppm.

### Comparative Example 8

An antibody was purified in a manner similar to the method in Example 10 except that Cellufine MAX Q-h (made by JNC Corporation) was used as the chromatography media. Collection efficiency of a monoclonal antibody was 101% and an amount of HCP in a collected fraction was 50 ppm.

### Comparative Example 9

An antibody was purified in a manner similar to the method in Example 10 except that Capto Q (made by GE Healthcare) was used as the chromatography media. Collection efficiency of a monoclonal antibody was 100% and an amount of HCP in a collected fraction was 52 ppm.

### Example 12

### (i) Chromatography Media and System

Media: media C

Column: inner diameter 0.5 cm, height 2.5 cm

System: Akta avant 25

### (ii) Antibody Solution and Solutions Used for Purification

Antibody solution: antibody solution e

A5 buffer: 20 mM Tris-HCl buffer solution (pH 7.5) 1 M NaCl aqueous solution

0.5 M sodium hydroxide aqueous solution

(iii) Procedures

The media was packed into the column up to a height of 5.0 cm. The column was connected to the system, and the A5 buffer by a volume of 10 columns was passed through the column at 0.49 mL/min to equilibrate the column. Then, 5.5 mL of the antibody solution was passed through the column at 0.25 mL/min. Then, the A5 buffer by a volume of 10 columns was passed through the column at 0.49 mL/min to wash the column. Then, the 1 M NaCl aqueous solution by a volume of 10 columns was passed through the column at 0.075 mL/min. Then, the 0.5 M sodium hydroxide aqueous solution by a volume of 5 columns was passed through the column at 0.075 mL/min to wash the column. Finally, the A5 buffer by a volume of 15 columns was passed through the column at 1.0 mL/min to reequilibrate the column.

A total amount of 5.5 mL of liquid passed through the column during passing the antibody solution therethrough, and 2.5 mL of a column washing liquid during washing of unadsorbed material were combined, and the resulting mixture was taken as a collected fraction. Collection efficiency of a monoclonal antibody (a proportion of an amount of the antibody collected therein to an amount of the antibody loaded on the chromatography media) was 96%, and an amount of HCP in the collected fraction was 16 ppm.

### Example 13

### (i) Chromatography Media and System

Media: media C

Column: inner diameter 0.5 cm, height 2.5 cm

System: Akta avant 25

### (ii) Antibody Solution and Solutions Used for Purification

Antibody solution: antibody solution h

A7 buffer: 20 mM Tris-HCl buffer solution+NaCl (pH 7.0, 22 mS/cm)

1 M NaCl aqueous solution

1 M sodium hydroxide aqueous solution

(iii) Procedures

The media was packed into the column up to a height of 5.0 cm. The column was connected to the system, and the A7 buffer by a volume of 10 columns was passed through the column at 0.245 mL/min to equilibrate the column. Then, 17 mL of the antibody solution was passed through the column at 0.245 mL/min. Then, the A7 buffer by a volume of 10 columns was passed through the column at 0.245 mL/min to wash the column. Then, the 1 M NaCl aqueous solution by a volume of 5 columns was passed through the column at 0.245 mL/min. Then, the 1 M sodium hydroxide aqueous solution by a volume of 5 columns was passed through the column at 0.245 mL/min to wash the column. Finally, the A7 buffer by a volume of 10 columns was passed through the column at 0.245 mL/min to reequilibrate the column.

A total amount of 17 mL of liquid passed through the column during passing the antibody solution therethrough, and 4.9 mL of a column washing liquid during washing of unadsorbed material were combined, and the resulting mixture was taken as a collected fraction. Collection efficiency of a monoclonal antibody (a proportion of an amount of the antibody collected therein to an amount of the antibody loaded on the chromatography media) was 93%, and an amount of HCP in the collected fraction was 2 ppm.

### Reference Example 1

### (i) Chromatography Media and System

Media: media C

Column: inner diameter 0.5 cm, height 5.0 cm

System: Akta avant 25

(ii) Antibody solution and solutions used for purification

Antibody solution: antibody solution f

A6 buffer: 20 mM sodium phosphate buffer solution (pH 6.5)

1 M NaCl aqueous solution

0.5 M sodium hydroxide aqueous solution

(iii) Procedures

The media was packed into the column up to a height of 5.0 cm. The column was connected to the system, and the A6 buffer by a volume of 10 columns was passed through the column at 1.0 mL/min to equilibrate the column. Then, 20 mL of the antibody solution was passed through the column at 0.25 mL/min. Then, the A6 buffer by a volume of 5 columns was passed through the column at 0.25 mL/min to wash the column. Then, the 1 M NaCl aqueous solution by a volume of 5 columns was passed through the column at 1.0 mL/min. Then, the 0.5 M sodium hydroxide aqueous solution by a volume of 5 columns was passed through the column at 0.25 mL/min to wash the column. Finally, the A6 buffer by a volume of 20 columns was passed through the column at 1.0 mL/min to reequilibrate the column.

A total amount of 20 mL of liquid passed through the column during passing the antibody solution therethrough, and 4.9 mL of a column washing liquid during washing of unadsorbed material were combined, and the resulting mixture was taken as a collected fraction. Collection efficiency of the antibody (a proportion of an amount of the antibody collected therein to an amount of the antibody loaded on the chromatography media) was 87%, and purity of a monomer in the collected fraction was 89.8%.

### 4. Analysis Method

### (1) Ion Exchange Capacity

To a 50 mL Erlenmeyer flask, 1 mL of chromatography media (chromatography media in an amount forming 1 mL in a volume upon packing the media into a column in a wet gel state) was put. Thereto, 50 mL of a 0.01 mol/L hydrochloric acid aqueous solution was added, and the flask was gently shaken. After the flask was left to stand at room temperature for 1 hour, 10 mL of supernatant was weighed in a 50 mL beaker by a volumetric pipette. Thereto, a phenolphthalein solution was added, and the resulting solution was titrated with a 0.01 mol/L sodium hydroxide aqueous solution. An adsorption amount of hydrochloric acid was calculated from a titer to determine ion exchange capacity per 1 mL of the chromatography media.

### (2) Rate of Modification of an Amino Group

Ion exchange capacity was measured on the chromatography media before and after the amino group in the compound having the plurality of primary amino groups bonded to the base media was modified with the hydrophobic group (hereinafter, also referred to as “before modification with the hydrophobic group” and “after modification with the hydrophobic group”) by the method described above, respectively. The rate of modification of the amino group in the compound having the plurality of primary amino groups was calculated using values of the measured ion exchange capacity, and according to the following equation.

$$\begin{matrix}Rate of \\ modification \\ ( \% )\end{matrix} = \frac{( \begin{matrix}Ion exchange capacity \\ before modification with \\ hydrophobic group\end{matrix} ) - ( \begin{matrix}ion exchange capacity \\ after modification with \\ hydrophobic group\end{matrix} )}{( \begin{matrix}ion exchange capacity before modifiaction with \\ hydrophobic group\end{matrix} )} \times 100$$

### (3) Collection Efficiency (%) of a Monoclonal Antibody

Absorbance at a measurement wavelength of 280 nm was measured on each of the antibody solutions before purification and the collected fractions obtained (antibody solutions after purification) used in Examples 1 to 12 and Comparative Examples 1 to 9 with a spectrophotometer. Collection efficiency (%) was calculated by converting the measured value into an amount of the antibody using an absorption coefficient E1% of 1.40, and according to an expression: {(amount of antibody in collected fraction)/(amount of antibody in antibody solution before purification)}×100.

### (4) Amount of HCP

An amount of the HCP in the collected fraction in the preparation step of the antibody solution, and an amount of the HCP in the collected fraction obtained in Examples and Comparative Examples were measured by using an Elisa kit (Cygnus, F550). The amount of the HCP was expressed in terms of a concentration (ppm) by using the amount of the HCP obtained, and an amount of the monoclonal antibody calculated from the absorbance at a measurement wavelength of 280 nm, and according to an expression: {amount of HCP in collected fraction (ng)}/{amount of monoclonal antibody in collected fraction (mg)}.

### (5) Collection Efficiency (%) of a Polyclonal Antibody

Absorbance at a measurement wavelength of 280 nm was measured on each of the antibody solution (antibody fraction f) before purification and the collected fraction obtained (antibody solution after purification) used in Reference Example 1 with a spectrophotometer. Collection efficiency (%) was calculated by converting the measured value into an amount of the antibody using an absorption coefficient E1% of 1.35, and according to an expression: {(amount of antibody in collected fraction)/(amount of antibody in antibody solution before purification)}×100.

### (6) Size Exclusion Chromatography Analysis Method

Column: TSK gel SuperSW mAb HR (Tosoh Corporation)

System: Infinity 1200 (Agilent)

Mobile phase: 0.2 M sodium phosphate buffer solution+0.1 M sodium sulfate (pH 6.7)

Flow rate: 0.7 mL/min

Injection amount: 50 μL

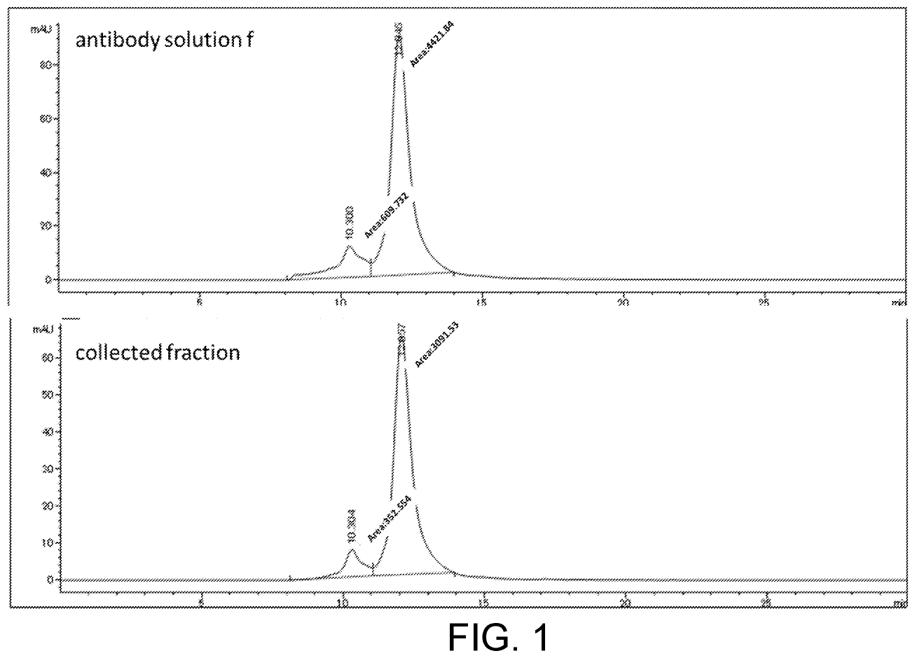

The antibody solution (antibody solution f) and the collected fraction used in Reference Example 1 were diluted 10 times with a mobile phase, and size exclusion chromatography analysis was performed. A chromatogram obtained is shown in FIG. 1. Peak areas of a monomer and an aggregate were determined from the chromatogram obtained, respectively, and purity of the monomer was calculated using the following calculating equation:

purity of monomer (%)=[(peak area of monomer)/{(peak area of monomer)+(peak area of aggregate)}]×100

The results obtained in Examples and Comparative Examples described above are summarized in Table 1.

TABLE 1

| | | | | Collection | |
|---|---|---|---|---|---|
| | | | Conductivity | efficiency | |
| | | | of antibody | of | |
| | | antibody | solution | antibody | HCP |
| | Media | solution | (mS/cm) | (%) | (ppm) |
| Example 1 | Media B | antibody | 6 | 96 | 22 |
| Example 2 | Media C | solution a | | 95 | 23 |
| Example 3 | Media D | | | 95 | 22 |
| Example 4 | Media E | | | 90 | 21 |
| Comparative | Cellufine | | | 98 | 114 |
| Example 1 | MAX Q-h | | | | |
| Comparative | Capto Q | | | 97 | 133 |
| Example 2 | | | | | |
| Comparative | Media A | | | 97 | 145 |
| Example 3 | | | | | |
| Example 5 | Media B | antibody | 14 | 97 | 35 |
| Example 6 | Media C | solution b | | 96 | 22 |
| Example 7 | Media D | | | 95 | 42 |
| Example 8 | Media E | | | 91 | 19 |
| Comparative | Cellufine | | | 97 | 147 |
| Example 4 | MAX Q-h | | | | |
| Comparative | Media A | | | 99 | 145 |
| Example 5 | | | | | |
| Example 9 | Media C | antibody | 6 | 95 | 3 |
| Comparative | Cellufine | solution c | | 97 | 22 |
| Example 6 | MAX Q-h | | | | |
| Comparative | Capto Q | | | 96 | 27 |
| Example 7 | | | | | |
| Example 10 | Media B | antibody | 14 | 96 | 8 |
| Example 11 | Media C | solution d | | 94 | 5 |
| Comparative | Cellufine | | | 101 | 5 |
| Example 8 | MAX Q-h | | | | |
| Comparative | Capto Q | | | 100 | 52 |
| Example 9 | | | | | |
| Example 12 | Media C | antibody | 22 | 96 | 16 |
| | | solution e | | | |
| Example 13 | Media C | antibody | 22 | 93 | 2 |
| | | solution h | | | |

Table 1 shows that high collection efficiency of the antibody is obtained and an amount of the impurities (HCP) in the collected fraction is small in Examples 1 to 13 in which the purification method according to the embodiment was used. Moreover, in Examples 1 to 13, regardless of the electrical conductivity levels of the antibody solution, the high collection efficiency of the antibody and a low amount of the HCP were able to be achieved. Further, also when antibody solutions a and b in which a citrate ion being a polyanion existed were used (Examples 1 to 8), the high collection efficiency of the antibody and the low amount of the impurities were able to be achieved.

Meanwhile, according to Comparative Examples in which any other media was used, a large amount of the HCP tends to be contained in the collected fraction, and the amount of the HCP was influenced by the electrical conductivity of the antibody solution and existence of the polyanion in the antibody solution. In Comparative Examples, when the electrical conductivity of the antibody solution was high (Comparative Examples 4, 5, 8 and 9), and when the polyanion existed in the antibody solution (Comparative Examples 1 to 5), the amount of the HCP particularly increased.

Antibody solution c has a solution composition in which the HCP was easily removed, and also when the solution was used, Example 7 resulted in a content of the HCP at a level one order of magnitude lower in comparison with Comparative Examples 6 and 7.

Antibody solution h is an antibody solution obtained by using a cation exchange media to purify antibody solution g in a flow-through mode before purification using media C. Example 13 in which such antibody solution h was used resulted in a lower content of the HCP in comparison with Example 12 in which antibody solution g without cation exchange chromatography was used.

Moreover, from the result in Reference Example 1, the aggregate as the impurities contained in the polyclonal antibody was able to be decreased by using media C in the flow-through mode, and the purity of the antibody was able to be improved from 84.5% to 89.8%.

Several embodiments of the invention are described, but the embodiments are presented as examples, and not intended to limit the scope of the invention. The new embodiments described herein may be embodied in various other forms, without departing from the scope of the invention, various omissions, substitutions and alternations can be made. The embodiments and their modifications fall within the scope and spirit of the invention, and are included in the invention as described in the appended claims and in the scope of their equivalents.

1. An antibody purification method, comprising bringing a solution containing an antibody and host cell protein into contact with a chromatography media to separate the antibody and the host cell protein,
wherein the chromatography media contains a base media containing porous particles, and a compound having a plurality of primary amino groups bonded to the base media, and 20 to 55% of the primary amino groups in the compound having the plurality of primary amino groups are modified with a hydrophobic group, and
collection efficiency of the antibody is 85% or more, and an amount of the host cell protein in an antibody solution after purification is less than 45 ppm.

2. The method according to claim 1, wherein the method is performed in a flow-through mode.

3. The method according to claim 1, wherein
more than 40% of primary amino groups in the compound having the plurality of primary amino groups are modified with the hydrophobic group.

4. The method according to claim 1, wherein the compound having the plurality of primary amino groups is selected from the group of polyallylamine, polyvinylamine, chitosan, polylysine, polyguanidine and polyornithine.

5. The method according to claim 4, wherein the compound having the plurality of primary amino groups is polyallylamine.

6. The method according to claim 5, wherein weight average molecular weight of the polyallylamine is 5,000 to 15,000.

7. The method according to claim 1, wherein the hydrophobic group has any structure represented by general formulas (1) to (3): 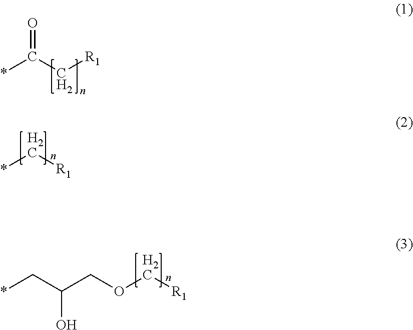
wherein,
n is an integer from 0 to 8,
R₁ is a phenyl group when n is an integer from 0 to 3, and H or a phenyl group when n is an integer from 4 to 8, and
an asterisk (*) represents a bonding site with a primary amino group in the compound having the plurality of primary amino groups.

8. The method according to claim 7, wherein the hydrophobic group has a structure represented by general formula (1).

9. The method according to claim 8, wherein, in general formula (1), n is an integer from 4 to 8, and R₁ is H.

10. The method according to claim 8, wherein, in general formula (1), n is an integer from 0 to 8, and R₁ is a phenyl group.

11. The method according to claim 1, wherein the hydrophobic group is a group derived from a compound selected from the group of valeric anhydride, caproic anhydride, enanthic anhydride, caprylic anhydride, pelargonic anhydride, benzoic anhydride, butyl glycidyl ether and phenyl glycidyl ether.

12. The method according to claim 11, wherein the hydrophobic group is a group derived from valeric anhydride or benzoic anhydride.

13. The method according to claim 1, wherein electrical conductivity of the solution containing the antibody and the host cell protein is 22 mS/cm or less.

14. The method according to claim 1, wherein a polyanion is further contained in the solution containing the antibody and the host cell protein.

15. The method according to claim 14, wherein the polyanion is one or more types selected from the group of a citrate ion, a phosphate ion and a sulfate ion.

16. The method according to claim 1, wherein the antibody is a monoclonal antibody.
